(19) United States
(12) Patent Application Publication
(10) Pub. No.: US 2005/0125040 A1
(43) Pub. Date: Jun. 9, 2005

(54) Method and apparatus for treating viral diseases

(76) Inventor: Peter Lathrop, San Diego, CA (US)

Correspondence Address: CATALYST LAW GROUP, APC, 4330 LA JOLLA VILLAGE DRIVE SUITE 220, SAN DIEGO, CA, 92122, US

(21) Appl. No.: 11/009,888

(22) Filed: Dec. 9, 2004

Related U.S. Application Data
(60) Provisional application No. 60/528,039, filed on Dec. 9, 2003.

(51) Int. Cl. A61N 1/00

(52) U.S. Cl. 607/2

(57) ABSTRACT

The present invention provides a miniature electrotherapy device having a housing and an embedded electronics module. The electronics module further comprises a preprogrammed microprocessor that is electrically connected to an electrode set for the delivery of optimal electrotherapy to treat viral outbreaks. The microprocessor adjusts the Waveform Parameters at the precise time and to the precise degree for delivering an optimal electrotherapy. Also included is a method for treating viral outbreaks using the invention device. This electrotherapy treatment can be interferential.

### RELATED APPLICATION

Benefit of priority under 35 U.S.C. 119(e) is claimed herein to U.S. Provisional Application No. 60/528,039, filed Dec. 9, 2003. The disclosure of the above referenced application is incorporated by reference in its entirety herein.

### FIELD OF THE INVENTION

The invention relates to a method and apparatus for delivering electrical stimulation to pathological tissue, and more particularly, to treating viral infections by applying an electrotherapy having a predefined and pre-programmed variation of Waveform Parameters for the optimal treatment of the affected skin or mucosa.

### BACKGROUND OF THE INVENTION

Clinical electrotherapy devices are used to implement many different types of human medical therapy protocols. Electrotherapy devices may be used to stimulate nerve cells and other forms of tissues in the human body to achieve a large number of therapeutic ends. In addition, electrical impulses cause muscles to contract and may be used for various forms of exercise and pain management. These electrotherapy devices are generally referred to as Transcutaneous Electrical Nerve Stimulator (TENS) devices, or TENS-like devices.

TENS and other microcurrent and/or millicurrent electrotherapy stimulation devices have been used successfully for the symptomatic relief and management of chronic intractable pain and other disorders for many years. TENS has also been used to promote healing via reduction of inflammation and the appropriate release of biochemicals, and to treat or maintain disorders including, but not limited to, viral infections such as herpes.

One effective TENS-based treatment is interferential therapy, which is generally described in T. W. Wing, Interferential Therapy: How it Works and What's New, The Digests of Chiropractic Economics, May/June 1992. In general, interferential therapy delivers at least two separate electrical currents from at least two pair of electrodes firing in synchronization. The delivered currents are of a high frequency, thus they are capable of painlessly penetrating the treatment site barrier (e.g., the skin). The currents are delivered in such a manner that they intersect, thereby forming a “beat frequency” at the point of intersection. Said beat frequency is generally comprised of a sum and a difference frequency and the difference frequency, or lower frequency, is an electrically stronger and more effective as a treatment. It is the current state of the art that interferential therapy is generally reserved for inpatient treatments, wherein a highly skilled therapist arranges four or eight electrodes and manually adjusts a TENS unit to deliver interferential therapy.

As stated above, electrical current or TENS-based therapies are useful for the treatment of herpes and other viruses. Viruses are small infectious agents containing a molecule of nucleic acid (RNA or DNA) as their genome. The viral nucleic acid is enclosed in a protein shell. The viral nucleic acid contains information necessary for programming the infected host cell to synthesize the specific number of macromolecules. Toward the end of the virus replicative cycle, more viral nucleic acids and coat proteins are produced. The coat proteins assemble together to form the symmetrical protein shell which encloses the nucleic acid genome.

There are eight identified herpes viruses that have been associated with human disease conditions. The alpha-herpes viruses, HSV-1, HSV-2, and VZV-2, known as oral herpes, genital herpes, and herpes zoster respectively, are neurotropic because they actively infect nervous tissue. Five other herpes viruses are lymphotropic because they replicate in the lymphatic system. These include HCMV (human cytomegalovirus), HHV-6, HHV-7, HHV-8 (KHSV) and EBV. HHV-6 has been associated with multiple sclerosis. HHV-8 (KHSV) and EBV have been linked to the human cancers Kaposi's Sarcoma and Epstein-Barr disease.

Disease states are also caused by a variety of other viruses. Viral hepatitis is a serious liver disease of particular concern for healthcare professionals. One form of hepatitis, hepatitis C, is considered responsible for approximately 10,000 deaths per year. The human papilomavirus (HPV) is responsible for most of the cervical cancers worldwide, genital warts and the formation of verrucae, warts that form on the soles of the feet. HPV has also been associated with several oral cancers.

### Oral Herpes: The HSV-1 Virus

Herpes simplex virus (HSV) infections of the oral tissues are among the most common infectious illnesses involving man. Both primary (initial) and recurrent forms of this infection occur, these being referred to as acute primary herpetic gingivo stomatitis, and recurrent herpes labialis. Although oral herpes infections may be considered primarily nuisance diseases, gingivostomatitis can be a very painful and debilitating illness. Furthermore, recurrent oral herpes in immunosuppressed subjects can be life-threatening (Overall. 1979: Ho, 1979: Faden et al. 1977).

The vast majority of oral herpes infections are caused by the HSV type 1 strain. Several factors contribute to the significance of oral herpes infections. First, herpes gingivostomatitis can be a severe illness. Fever, toxicity, and exquisitely painful mouth lesions may interfere with fluid intake and require hospitalization for intravenous fluids. Second, frequent recurrent lesions of the lips are of cosmetic concern, particularly in females. Third, cold sores may be the source of HSV for transmission to immunosuppressed or other hospitalized patients. Fourth, oral herpes in the immunosuppressed patient is often a severe, life-threatening disease. Finally, there is currently no satisfactory and fully effective form of therapy for either primary or recurrent mucocutaneous HSV disease in the normal host.

Most patients hosting a herpes virus infection develop vesicles within 12 hours, which rupture to form ulcers or crusts in 36 to 48 hours. Most patients lose the crust and have healed ulcers by day 8 to 10. Results from clinical trials on recurrent herpes labialis has shown that about 25% of patients had episodes one or more times a month, almost two-thirds had one episode every 2-4 months, and less than 25% had an episode less often than every 4 months (Spraunce et al, 1977).

### Genital Herpes: The HSV-2 Virus

After inoculation and limited replication at genital sites, HSV-2 ascends along neuronal axons to establish latent infection in the lumbosacral ganglia. During this initial phase, infectious virus is present at genital sites for days or weeks, usually without lesions. When a new cycle of viral replication is triggered, reactivation occurs and infectious virus is delivered back down the neural pathways to the mucosa or skin. The return of infectious virus to genital sites during HSV-2 reactivation rarely causes any symptoms. HSV-2 is a chronic, persistent infection that causes subclinical reaction in about 1% of infected persons. Since about 45-50 million people in the U.S. are infected, HSV-2 can spread efficiently and silently through the population. People who have sexual contact with many partners will frequently have exposure to an infected person who is shedding HSV-2. As the overall prevalence of HSV-2 infection continues to rise, contact with fewer partners will permit exposure.

The concept that HSV persists in the nuclei of cells in the sensory ganglia suggests that any topical treatment will not be effective in destroying the virus in these hidden locations. A variety of treatments have been used for genital herpes but none is entirely satisfactory. To date, no satisfactory vaccine has been found.

### Herpes Zoster: Varicella-Zoster (VZ)

Herpes zoster, also known as shingles, is due to invasion of posterior root ganglia by the causative virus and is characterized by severe pain followed by a rash over cutaneous distribution of the affected nerve. VZ causes two diseases, varicella (chickenpox) resulting from the first exposure to the virus in childhood, and zoster, a secondary infection due to reactivation of the latent VZ virus. Shingles is a painful and potentially debilitating disease caused by a reactivation of the varicella-zoster virus, the same herpes virus which causes chickenpox. A major challenge for physicians in managing patients with shingles is alleviating the severe pain associated with an active shingles rash, as well as postherpetic neuralgia (long-term debilitating pain) which may occur following rash healing.

### Herpes and Multiple Sclerosis

A strain of reactivated herpes virus may be associated with multiple sclerosis (MS), an autoimmune disorder in which the body attacks its own tissues. Results of a study conducted by scientists at the National Institute of Neurological Disorders and Stroke (NINDS) in Bethesda, Md., add to mounting evidence of the role of viral triggers in MS and may serve as the cornerstone for clinical trials using antiherpetic agents as a treatment. In the study, more than 70 percent of patients with the relapsing-remitting form of MS showed an increased immune response to human herpes virus-6 (HHV-6) and approximately 35 percent of all MS patients studied had detectable levels of active HHV-6 in their serum.

### Human Papillomavirus

Human papillomavirus (HPV) is one of the most common sexually transmitted diseases. Genital HPV infections are widespread among sexually active adults. Genital warts often occur in clusters, and can be very tiny or can occur in large masses. Treatment includes the application of trichloracetic acid or podophyllin solution. Warts can be removed by cryosurgery, electrocautery or surgery. Although elimination of the warts is possible, the viral infection persists and warts often reappear after treatment.

### Electrical Stimulation-Based Virus Treatments

To date, there are very few satisfactory treatments, vaccines, or cures for viral infection. Drug treatments, either topical or ingested, have shown generally limited benefits. As an alternative to the pharmaceutical approach, the electrical stimulation of infected tissues has been explored. These methods involve the application of electrodes to the skin near the infected region.

U.S. Pat. No. 4,913,148 issued to Diethelm, claims a method for accelerating the healing process of a herpes outbreak using two conducting electrodes to deliver an electrical current. The electrical current delivery device is programmed using a keyboard to deliver a monopolar pulse in the form of a square wave having a constant frequency of 30 Hz and lasting about 0.2 msec. Via the keyboard, an operator can shift the frequency parameter during the course of treatment to increase or diminish the current frequency. The device cannot be pre-programmed to deliver a treatment having varied parameters, and thus requires a skilled operator to vary the parameters using the keyboard. Real-time parameter variance is subject to variations in timing and degree for the parameter shifts from treatment to treatment.

U.S. Pat. No. 5,133,352 issued to Lathrop et al., claims a treatment method using an electrical device comprising two electrodes and an electrical power source that delivers from a 9 volt battery a single and unvarying low DC voltage. The claimed method for using the device states that the device is placed on the human body in the area surrounding a lesion and the low DC voltage is delivered to the contact point for a time period under one minute, at which point delivery of the voltage is ceased, and then reapplied again before two hours has passed. The cycle is repeated for a maximum 8 hour time span. The method of this invention discloses a directional current treatment voltage that is incapable of variance in its waveform parameters.

U.S. Pat. No. 5,607,461 also issued to Lathrop, claims a device for applying electrical stimulation to a lesion. The device comprises two removable electrodes, connected to a circuit having a battery and operated by a manual depression switch. The circuit is disclosed as electrically coupling the battery to the electrodes via two lead wires. In addition, the circuit is capable of operating a visual indicator, which details the operational status of the invention. When the switch is depressed to activate the electrical circuitry, an LED is illuminated indicating whether there is proper current flow in the device. The device delivers a single, non-variable directional current wherein the parameters cannot be varied.

U.S. Pat. No. 6,083,250 also issued to Lathrop, claims a method for preventing herpes lesions using an electrical device comprising two electrodes and an electrical circuit having a manual power switch, a controller/pulse generator and a 9 volt battery. The method of treatment includes: placing the device's pair of electrodes in proximity to the lesion; manually closing the switch; delivering an alternating current to the electrodes. The parameters for the electrical energy delivered from the device can be variable in that it is disclosed that the controller can be pre-programmed to deliver 9, 18 or 27 milliamps, can be preprogrammed to deliver a frequency between 1 Hz and 600 Hz, and can be preprogrammed to deliver a square, modified biphasic square or sine wave pulse. However, the pre-programmed parameters of this invention remain static following programming, and the treatment must be stopped and the parameters manually changed before a second varied parameter set can be delivered. This is neither an efficient treatment, nor a simplified method for home use by the patient.

U.S. Pat. No. 6,594,527 B2 issued to Mo, claims an electrical stimulation device having an independent module and housing. The housing comprises an LCD display screen, and two separate battery operated circuits. The housing's first circuit is used to generate a series of alternating monopolar bursts. Said monopolar bursts are provided to each individual therapy device by a prescribing physician, each device being varied form another in milliamps, microvolts, current profile, waveform and number and duration of treatments available. The second circuit operated the devices LCD visual display, for displaying to the user the physician prescribed protocol for the user to follow during treatment. The separate module comprises two electrodes, and a connection port for connecting with the housing. Because of the difficulties with patient compliance and lack of training for the lay-person, it is undesirable to have a device wherein the user adjusts the electrical parameters according to a set of programmed physician's orders that are displayed on the device. Additionally, it is inefficient that the physician's instructions for the parameter variation are displayed on the device's LCD, which, when in use, is out of the view of the user, thus making the likelihood of missing an instruction highly probable.

U.S. Pat. No. 6,618,652 B1 issued to Silverstone, claims an electrical stimulation device wherein the device comprises: a hemispherical electrode having an equatorial line dividing the first electrode form the second; a disposable cartridge comprising said electrodes, an electric circuit and a battery; and a display to notify the user of how many pulses have been delivered. There is no disclosure of parameter variability in this application.

Although electrotherapy treatment of viral disorders shows promise, the current state of the art is deficient in delivering an optimal therapy. Thus there is a need in the art for an improved device and method for the treatment of viruses using electrical stimulation. Said need includes, but is not limited to the need for delivery of an optimized electrical current profile that: does not require monitoring and adjustment by the user (lay-person and skilled user); is not limited to a pre-set, non-varying electrical stimulation protocol; does not rely on the intervention of a user (whether lay or skilled) to adjust parameters during treatment; and that delivers a painless, yet strong electrical current for the treatment of viral infections.

### BRIEF SUMMARY OF THE INVENTION

Generally, the present invention provides a miniature electrotherapy unit including a housing and an electronics module embedded within said housing. The electronics module further comprises a power source that is electrically connected to a preprogrammed microprocessor with memory (hereinafter “pre-programmed microprocessor”) that is electrically connected to an electrode set. The electrode set is comprised of at least one electrode pair. Said electrodes will traverse said housing and are minimally exposed outside of said housing for the delivery of electrotherapy based treatments.

The pre-programmed microprocessor is capable of delivering an optimal electrotherapy treatment regime, wherein sequential pulses form a plurality of waveforms available for specific clinical needs. As used herein, Waveform Parameters include, but are not limited to pulse frequency, pulse amplitude, pulse width, duration, modulation, current, pulse times and intensity. Waveform Parameters are pre-programmed into the microprocessor based on a predetermined optimal treatment regimen for treating a viral infection.

In the prior art of electrotherapy, the electrical energy parameters (amperage and frequency) are manually adjusted during therapy. Depending on which treatment is being delivered, these electrical energy parameters are adjusted at specific times, thereby delivering a changed current from time point A to time point B to time point C . . . etc. However, in order to optimally deliver these variations in an electrotherapy treatment, it is desirable to change these and other parameters (collectively, the Waveform Parameters) at a precisely accurate time and degree. Optimization is lost in the prior art via the reliance on an operator's intervention to make these mid treatment adjustments. To overcome these deficiencies, the current invention has the changes in Waveform Parameters and the critical time points pre-programmed into a microprocessor. Thus the delivery of an electrical treatment is optimally efficient in that the degree of change and the timing for change is precise. Furthermore, the error factor introduced by manual adjustment, and that is increased when the adjustments are made by a non-skilled user, have been eliminated. Thus the current invention delivers an optimal electrotherapy treatment and is an improvement over the prior art. Variations of Waveform Parameter settings and time points for making these variations during an electrotherapy treatment are known in the art.

To achieve the improvement, the current invention device comprises a microprocessor with memory. The microprocessor's memory is programmed to hold the optimal Waveform Parameters, and to precisely deliver instructions as to when to deliver, alter and otherwise vary these Waveform Parameters during a treatment. The electrical circuitry will deliver either standard therapy treatment Waveform Parameters using a single pair of electrodes, or will deliver interferential therapy Waveform Parameters using at least two pairs of electrodes. Other therapies can also be programmed into the pre-programmed microprocessor of the current invention.

Adjustments in said Waveform Parameters using the current invention occur without the intervention of a user, and thus are precise in both timing and in degree of parameter settings. By way of the pre-programmed microprocessor, the inherent errors and time lapse variation associated with user or therapist operation is eliminated. The Waveform Parameters of the current invention are determined, delivered and changed by command of the microprocessor in a cycle that has been pre-programmed into said microprocessor in accordance with known optimal Waveform Parameters in the electrotherapy arts for treating viruses. Pre-programming the treatment Waveform Parameter cycles into the microprocessor allows the microprocessor to deliver precise and time critical electrotherapy without interference by a user who would otherwise have to adjust the unit.

The electrodes forming the electrode set of the current invention are of a size and length to effectively deliver the electrical treatment to the treatment area. Most users of a herpes treatment device would like to be discrete, thereby avoiding the stigma associated with having herpes. For this reason, it is preferable that the electrodes of the current invention are as small as functionally possible. For treatment of oral herpes and herpes zoster, which occur on the lips, torso, arms and cranium, treatment may occur in public. Thus both the device and the electrodes are very small, allowing for a discrete public use. On the other hand, treatment of herpes and other viral infections on the genitals and anus will require longer electrodes in order to effectively reach and treat outbreaks in the vagina or around the body at the anus. These types of treatments are not performed in public, and thus the longer electrodes do not pose any threat to user privacy.

As mentioned above, the electrode set of the current invention may comprise a single pair of electrodes for delivery of non-interferential therapy, or may comprise at least two pair of electrodes for the delivery of interferential therapy. For interferential therapy using two pair of electrodes, the electrode pairs will be arranged such that the anode of one pair is diagonal to its cathode, and the anode and cathode of the second pair are similarly placed. Those of ordinary skill in the art will recognize such configuration as the “criss-cross” pattern. For embodiments wherein more than two pairs of electrodes are used to deliver an interferential therapy, the same principle applies, such that each anode/cathode pair is spaced at the maximum longitudinal distance across the circumference of electrode pairs, thereby forming a “star” pattern. Furthermore, the electrodes are sufficiently insulated to prevent any premature electrical arc of current between neighboring electrodes.

### BRIEF DESCRIPTION OF THE FIGURES

FIG. 1A is a top view of the electrotherapy device showing the housing with recessed section, the electronics module and its components, and the minimally protruding electrodes.

FIG. 1B is a front view of the electrotherapy device showing minimally protruding electrodes at the recessed section of the housing.

FIG. 1C is an image of the electrotherapy device in comparison to a user's hand thereby showing one example of how the electrotherapy device can be small and discreet.

FIG. 2A is a top view of the electrotherapy device showing the housing with recessed section, the electronics module and its components, and the minimally protruding electrodes used for treating hard to reach treatment areas.

FIG. 2B is a front view of the electrotherapy device showing one embodiment of the minimally protruding electrodes when used for treating hard to reach treatment areas.

FIG. 2c is a front view of the electrotherapy device showing an extended housing component with minimally protruding electrodes used for treating hard to reach treatment areas.

FIG. 3A is a cross sectional view of an electrode wrapped in an insulating material to prevent premature arcing.

FIG. 3B is a cross sectional view of a plurality of electrodes wrapped in a single insulating material to prevent premature arcing.

FIG. 3B is a front view of an electrotherapy device wherein two pair of electrodes are used to perform interferential therapy.

FIG. 4A is an illustration of a gap used in the current invention to activate the electrotherapy device wherein said gap is between the anode and the cathode electrodes.

FIG. 4B is an illustration of a gap wire used in the current invention to activate the electrotherapy device.

### DETAILED DESCRIPTION OF THE INVENTION

Device

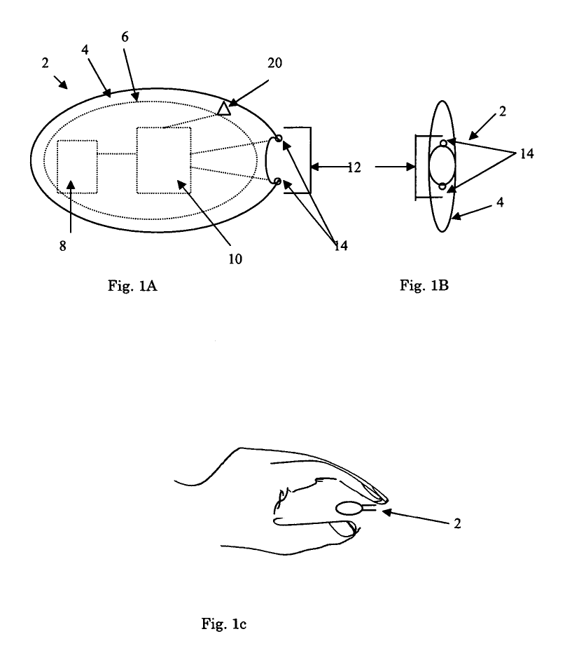

Turning now to the drawings, in which like numerals indicate like elements throughout the several views, FIGS. 1a and 1b show that the current invention is directed towards an electrotherapy device 2 that is small and unobtrusive, thereby allowing the user to comfortably and discretely receive electrotherapy-driven treatments for viral infection and associated symptoms. The electrotherapy device 2 of the current invention at least comprises a housing 4 and an electronics module 6. The electronics module further comprises a power source 8 that is electrically connected to a pre-programmed microprocessor with memory (hereinafter “pre-programmed microprocessor”) 10 that is electrically connected to an electrode set 12 (an electrode set, as used herein, is at least one pair of electrodes).

Housing 4 is made of an electrically non-conductive material and can be round, oval, square, rectangular, or of any shape suitable for embodying the current invention. In the preferred embodiment, housing 4 is oval in shape and is sized to be held discretely between the thumb and forefinger of the user's hand (FIG. 1c). By way of example only, the oval shaped housing 4 in this embodiment can have a large diameter of between 0.5 to 5.0 cm, most preferably 2.25 cm, and a short diameter of between 0.3 to 3.0 cm, more preferably 1.49 cm. FIG. 1a shows the oval surface being referred to in this example of the preferred embodiment. Further, in this example, oval shaped housing 4 can have a thickness of between 0.1 and 1.0 cm, most preferably 0.5 cm. FIG. 1b shows the thickness being referred to in this example of the preferred embodiment. Also shown in FIGS. 1a and 1b, exposed at one end of the housing 4 is electrode set 12. It is notable that the electrotherapy device 2 of the current invention can be of any size and shape. While smaller sizes are preferred because of the ease of use and discreetness that is offered with a smaller size, larger electrotherapy devices 2 are well within the spirit of the current invention.

An individual electrode 14 forming electrode set 12 can be of a variety of sizes. Preferably, for use in treating oral herpes, shingles or other viral complications that are easily within reach on a user, each individual electrode 14 is exposed about 2 mm outside of housing 4. In this embodiment, one end of housing 4 is shaped such that the 2 mm of exposed electrode is raised above the surface of said housing 4. As used with the oval housing design, one end of the oval shape is recessed to allow for adequate exposure of the individual electrodes 14. The recessed area can be concave, thereby forming a bowl-like shape with the electrodes raised up from the lip of the bowl. Alternatively, the recessed area can be substantially flat, again with the electrodes raised from the outer circumference of the recessed area. While electrodes 14 of electrode set 12 are described as being exposed approximately 2 mm outside of housing 4, such an exposure length is only preferable and is not a necessity. The length of the electrodes 14 of electrode set 12 can be of any length, and those of ordinary skill in the art will readily make the device of the current invention using a variety of electrode lengths. Such variations are well within the spirit of the current invention.

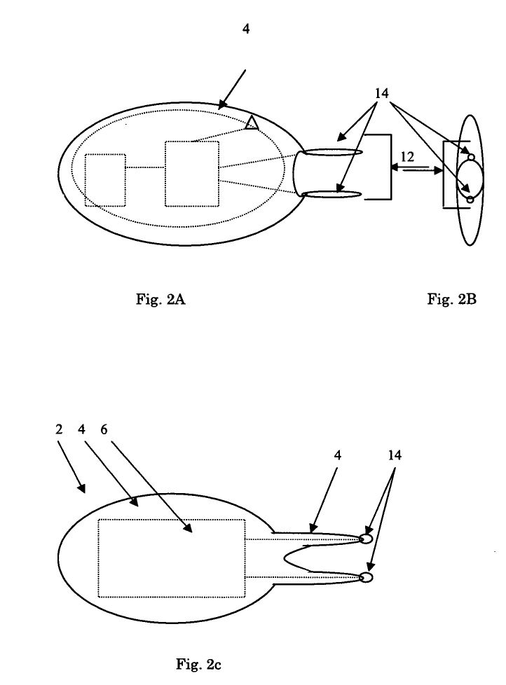

In an alternative embodiment, shown in FIGS. 2a and b, electrodes 14 of electrode set 12 are adequately sized to deliver treatment to areas of the user's body that are more difficult to reach. For example, herpes virus infections commonly occur within the vagina and/or near the anus. For treatment of these infections, it is desirable that the individual electrodes 14 are preferably, but not necessarily, exposed between 25-50 mm outside of housing 4, most preferably 38 mm. In this embodiment, the user is able to perform electrotherapy on these areas without having to uncomfortably contort their body to reach said treatment area. Furthermore, for use in treating female genital herpes outbreaks, the user need insert only these longer electrodes 14 into the infected area in order to perform treatment using the device of the current invention, whereas the shorter electrode 14 embodiment would require insertion of the all or part of the device as well.

In a further embodiment, shown in FIG. 2c, the added length of electronics device 2 useful for treatments in hard to reach areas is embodied in housing 4 of the electronics device 2. In this alternative embodiment, housing 4 is preferably, but not necessarily, extended between 23 mm and 48 mm with electrode set 12 preferably, but not necessarily, being exposed outside of said housing extensions an additional about 2 mm. Such an embodiment will add protection to electrode set 12 through the durability of housing 4. An alternative way to describe this same principle is that the electrode set 12 extends between 25 mm and 50 mm from the housing 4, and further comprises an electrically non-conductive sheathing covering between 23 mm and 48 mm of said electrode set 12, thereby leaving an approximate 2 mm of exposed electrode at the tip distal the housing. Those of ordinary skill in the art will readily make the electronics device of the current invention using a variety of electrode and housing sizes. Such variations are well within the spirit of the current invention.

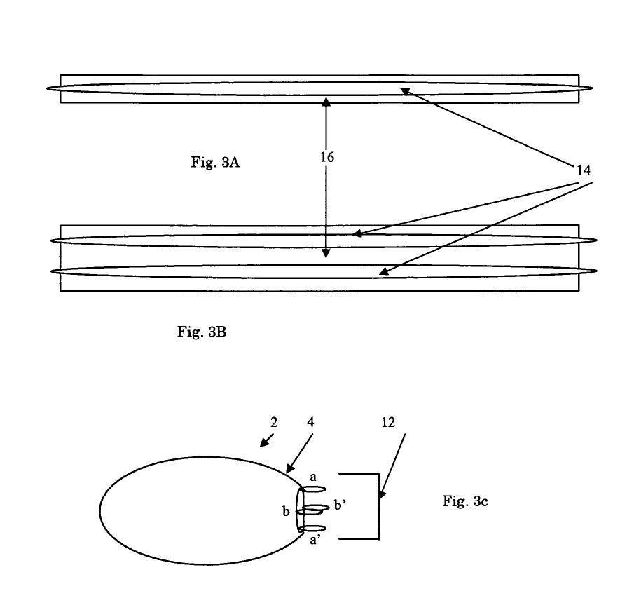

In a preferred embodiment, electrode set 12 comprises a single pair of individual electrodes 14, and more preferably comprises two pair of individual electrodes 14. Said individual electrodes 14 may be embodied as separate prongs, that, when used in correspondence with other individual electrodes 14, will appear as separate electrode shafts (FIG. 3a). In this embodiment, each separate electrode is well insulated with an electrically non-conductive material 16 to prevent an undesired and premature electric arc between the cathodes and the anode. Those of ordinary skill in the art are familiar with materials and methods for insulating electrodes.

In another embodiment, said individual electrodes 14 are embodied in a cluster (FIG. 3b). In this embodiment, said cluster comprises as a single unit both an electrically non-conductive material 16 and the electrically conductive individual electrodes 14. By way of example only, the electrode cluster in FIG. 3b shows a single pair of electrodes 14 embedded in a non-conductive material 16. In use, the individual electrodes 14 forming a pair are electrically considered an anode or a cathode, respectively. The anode/cathode determination for an individual electrode unit may be static (as is common in DC systems) or they may alternate (AC system). In a further embodiment, and particularly when the electrodes 14 are used for interferential therapy, said clusters may comprise four or more individual electrodes 14 (FIG. 3c). In this alternative embodiment each pair of electrodes will have an independent anode and a cathode, allowing each separate pair of electrodes to fire independently, but in synch with the other pair of electrodes. In FIG. 3c, the two pair of electrodes are configured in a criss cross pattern, with the first pair denoted as a and a′ and the second pair denoted as b and b′. Electrode arrangement and firing techniques for interferential therapy are well known in the art.

Turning now to FIG. 1a and the electronics module 6 of the electrotherapy device 2, it is shown that the electrode set 12 is electrically connected to a power source 8; and a pre-programmed microprocessor 10 for the delivery of electrotherapy. Power source 8, pre-programmed microprocessor 10, and a portion of electrode set 12 are preferably housed within the housing 4. In one embodiment, power source 8 is a switchable power supply, for example, such as a single pole dual throw switch (SPDT). In this embodiment, a user initiates electrotherapy via manual translocation of said SPDT whereby translocation closes the circuit to allow delivery of electrical current. Other manual power supplies are well known by those of ordinary skill in the art, and are readily substituted into the current invention.

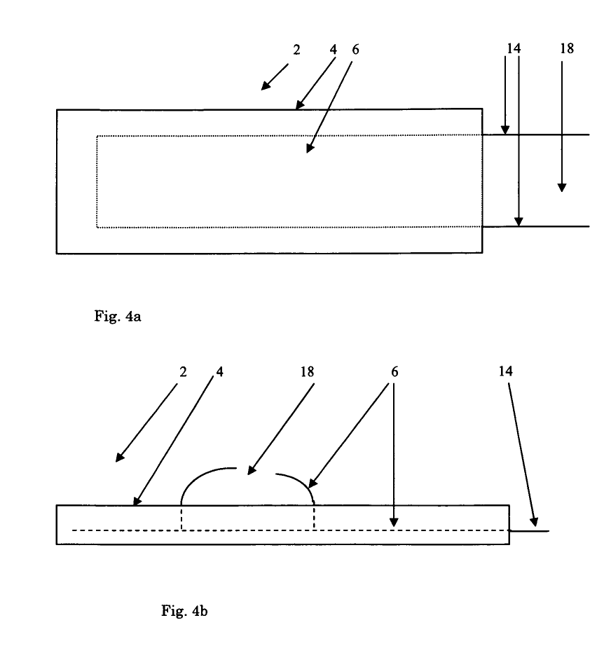

In a more preferred embodiment shown in FIG. 4a, power source 8 is activated by using the conductivity of the treatment surface (e.g., skin) to close a gapped circuit. In this alternative embodiment, an exposed portion of the electronics module 6 has an electrical gap 18. Preferably, this exposed gap 18 in electronics module 6 is between the electrodes 14 forming the electrode set 12; however, it may be a break anywhere in said circuit. For example, said gap 18 may present as an exposed and broken wire at the surface of housing 4 where the user's thumb or forefinger is placed to hold the electrotherapy device 2 during operation (FIG. 4b). When the electrotherapy device 2 is placed in contact with a patient's body (either the treatment area when using the electrodes as a gap 18 or the thumb/forefinger when using a gap wire 18), the circuit is closed by way of the electrical conductivity of said user's body. In this embodiment, the user's touch closes the electrical circuit of electronics module 6 and thus, electrical current begins flowing from power source 8 through said electronics module 6. Those of skill in the art will readily design gap circuits as anticipated by the current description of the current invention.

In a preferred embodiment, power source 8 is small enough to fit within housing 4, without causing housing 4 to loose its discrete sizing. Said power source 8 may be a battery, such as the family of lithium batteries known and readily available in the art; however, a variety of power sources are available for use with the current invention, and the use thereof does not exceed the scope of the current invention. Suitable power sources include, but are not limited to primary cell batteries, rechargeable batteries, embedded power sources and telemetric power. Current delivered from power source 8, and through electronics module 6 can be of any amperage however milliamperage (millicurrent) and microamperage (microcurrent) are more commonly employed.

Electrically coupled to the power source 8 is a pre-programmed microprocessor 10. In a preferred embodiment, pre-programmed microprocessor 10 is pre-programmed to deliver a series of currents having the optimal Waveform Parameters for treating viral infections. Pre-programmed microprocessor 10 is programmed to determine, deliver and change these Waveform Parameters in a cycle and range that is based on the profile of Waveform Parameter shifts known to optimally treat viral infections. This processor based shifting of Waveform Parameters, thereby removes the need to have a user manually adjust these same Waveform Parameters as prescribed in a given treatment protocol. Electrotherapy treatment protocols for the treatment of viruses, including the Waveform Parameters and cycle times, are well known by those of skill in the art. By removing user (both lay and skilled) facilitated adjustments of the Waveform Parameters from the treatment regimen, as is currently required in the art, the current invention is also necessarily removing inherent user errors, such as delays and inaccuracies in shifting from one set of Waveform Parameters to another. By way of the pre-programmed microprocessor, the device precisely adjusts the Waveform Parameters to the prescribed settings, and does so at the precisely optimal time, thus delivering an efficient and cycle of changing Waveform Parameters for the optimal treatment of viral infections. Optimal treatment of viral infections reduces the time and costs of treatment.

In the prior art of electrotherapy, the Waveform Parameters are manually adjusted during therapy, as is discussed above. Depending on which treatment is being delivered, the Waveform Parameters are adjusted to different degrees at specific times, thereby delivering a changed current from time point A to time point B to time point C . . . etc. Such Waveform Parameter settings and time points for changing are known in the art. However, in order to deliver the most optimal electrotherapy treatment, changes in the Waveform Parameters must be precisely accurate in both timing and settings. Optimization is lost in the prior art via reliance on an operator's intervention to make these mid treatment adjustments, wherein errors in time and degree are inherent. To overcome these deficiencies in the prior art, the current invention has the Waveform Parameters settings and the critical time points pre-programmed into a microprocessor in order to deliver the precise and optimal therapy as is known in the art.

The pre-programmed microprocessor's 10 memory is programmed to hold the optimal Waveform Parameters, and to precisely deliver instructions as to when to deliver, alter and otherwise vary the waveform parameters during a treatment. The electrical circuitry will deliver either standard therapy treatment Waveform Parameters using a single pair of electrodes 14 forming electrode set 12, or will deliver interferential therapy Waveform Parameters using two or more pairs of electrodes 14 forming electrode set 12. Other therapies can also be programmed into the pre-programmed microprocessor of the current invention.

The pre-programmed microprocessor 10 can be further programmed with a positive feedback loop that will determine resistance at the treatment surface, and will thereby adjust delivery of the Waveform Parameters to account for the resistance of the body. Each user will have a different resistance pattern, and thus will require a unique adjustment to overcome this resistance. Such adjustments will assure that the profile of the Waveform Parameters actually delivered to the treatment site is consistent with the desired profile for the Waveform Parameters.

More specifically for this embodiment, the treatment surface may pose a user specific resistance, which, if not accounted for, will affect the profile of the Waveform Parameter actually delivered to the treatment site. Using the positive feed back loop, the electrotherapy device 2 will detect resistance at the treatment site through the at least one electrode pair 12, will deliver data regarding said resistance to the pre-programmed microprocessor 10, which will then analyze said resistance and adjust the delivered profile for the Waveform Parameter allowing the actual received Waveform Parameter profile to remain consistent with the desired profile.

Because the electrical current delivered from electrotherapy device 2 is preferably an undetectable microcurrent, it is desirable to have an indicator device 20 (see FIG. 1) to alert the user as to when the treatment is occurring, when a treatment has ended and whether the power source is properly delivering electrical current. Such indication is preferably performed by an auditory cue; however, numerous other indication options exist within the scope of the current invention. In the preferred embodiment, indicator device 20 is an auditory device and is responsive to the power delivery generated by power source 8, thereby notifying the user of the on/off status of said electrotherapy device 2. Indicator device 20 is electrically connected to electronics module 6 on one end, and is capable of delivering a sound to the external surroundings of housing 4, thereby being heard by the user for the determination of the electrical current status of electrotherapy device 2. For example, the indicator device 20 chirps once when power supply 8 is in the on position, indicating to the user that a current is running through said system. When power supply 8 is in the off position, indicator device 20 is silent. Indicator device 20, also allows the user to monitor whether the electrotherapy device 2 is working properly, and when a treatment session has completed its course. By way of example only, indicator device 20 can be silent when no electrical current is flowing through electronics module 10. Upon activation of the electronics module 10 by a user, indicator device 20 will indicate a flow of current by chirping once, or will indicate no current flow by remaining silent. Once the activated electrotherapy device 2 has run its course of treatment indicator device 20 will indicate such by chirping twice. Other combinations and variations of sounds are readily apparent to those of skill in the art, given the current disclosure. Furthermore, visual indicators and other means for indicating on/off status and proper functioning are well known in the art and can be readily applied to the current invention.

The power generated by the electrotherapy device 2 when power supply 8 is activated is controlled by the pre-programmed microprocessor 10, which delivers optimal Waveform Parameters to the electrode set 12. Electrode set 12 is electrically connected to the electronics module 10, traverses housing 4 and is exposed outside of said housing 4. Said exposed end of electrode set 12 is placed in contact with a treatment surface. Electrodes and electrically conductive materials forming electrodes are well known to those of ordinary skill in the art.

Method of Use

One preferred method of use for electronics device 2 uses a single pair of electrodes 14 to form the electrode set 12 for the delivery of a non-interferential therapy. In this method of use, the user, sensing a herpes outbreak, will grasp electrotherapy device 2. The user will place the electrode pair of said electrotherapy device 2 on or near the herpes outbreak and will activate the electrotherapy protocol. The protocol is activated via closing the gap 18 in electronics module 6, which, in this example is done via placing the at least one electrode pair 12 to the conductive surface of the treatment area. Upon activation of the protocol, indicator device 20 will emit a single chirp to indicate proper functioning of electronics device 2. The treatment protocol is delivered to the treatment site, and the particular Waveform Parameters of said treatment protocol are determined and controlled by pre-programmed microprocessor 10. The Waveform Parameters of said treatment protocol are dynamic and will change during a treatment protocol. Said changes are based on the optimal Waveform Parameters for treating a viral infection; however, the improvement over the art is that the changes are made under the precise directions of pre-programmed microprocessor 10. Such precision in control and delivery are advantageous in that inherent errors in user intervention are eliminated, and, thus, treatment is more effective. Once the treatment protocol has run it course, the indicator device 20 will chirp twice, indicating to the user that the electrotherapy device can be removed from the treatment area until the next application is indicated.

In another preferred method of use, electrode set 12 is two pair of electrodes 14 configured to deliver an interferential therapy. In this method of use, the user, sensing a herpes outbreak, will grasp electrotherapy device 2, place the said electrotherapy device 2 on or near the herpes outbreak and will activate the electrotherapy protocol via by manually translocating a single pole dual throw switch. Upon activation of the protocol, indicator device 20 will emit a single chirp to indicate proper functioning of electronics device 2. The treatment protocol is delivered to the treatment site, and the particular Waveform Parameters of said treatment protocol are determined and controlled by pre-programmed microprocessor 10, as is described above. User resistance is accounted for and the proper adjustments made so that the delivered/received waveform parameters match the desired parameters. For the interferential therapy based treatment protocols, the pre-programmed microprocessor 10 is additionally programmed to deliver the treatment protocols to each pair of electrodes such that the electrode pairs deliver an electrical current that is synchronized to bisect at the treatment area. At the point of bisection, the individual currents will form a beat pulse or beat frequency. Those of ordinary skill in the art will readily employ this and other interferential therapy based treatment protocols in the inventive device, and such variations are within the spirit of the current invention. Again, as above, the electrical current is precisely controlled by the pre-programmed microprocessor 10, and thus is a more effective treatment that is available in the prior art. Once the treatment protocol has run it course, the indicator device 20 will chirp twice, indicating to the user that the electrotherapy device can be removed from the treatment area until the next application is indicated.

1. A pre-programmed microprocessor based electrotherapy device for the treatment of viral infections in a mammal comprising:
a. an optimally shaped and dimensioned housing further comprising an electrically non-conductive material; and
b. an electronics module further comprising at least:
i. an electrode set said electrode set being of an optimal size and configuration to deliver the electrotherapy treatment regimens of the current invention; and
ii. a microprocessor, said microprocessor being pre-programmed with a Waveform Parameters for delivering optimal electrotherapy treatment regimens.

2. The device of claim 1 wherein the optimal shape of the housing is an oval shape and the optimal dimensions of the housing are between 0.5 cm to 5.0 cm for the large diameter of the oval, between 0.3 cm and 3.0 cm for the short diameter of the oval, and between 0.1 cm and 1.0 cm for the thickness.

3. The device of claim 3 wherein the housing is an oval shape and the optimal dimensions of the housing are 2.25 cm for the large diameter of the oval, 1.49 cm for the small diameter of the oval, and 0.5 cm for the thickness.

4. The device of claim 1 wherein the electrode set comprises a single pair of electrodes.

5. The device of claim 4 wherein the two electrodes forming the single pair of electrodes each protrude from the device housing, said protrusion being between 2 mm and 50 mm.

6. The device of claim 5 wherein the two electrodes protrude 2 mm from the device housing and is useful for electrotherapy treatment of viral outbreaks at easily accessible areas of the body.

7. The device of claim 5 wherein the two electrodes protrude between 25 mm and 50 mm from the device housing.

8. The device of claim 7 wherein the two electrodes protrude between 25 mm and 50 mm from the device housing and further comprise an electrically non-conductive sheath covering between 23 mm and 48 mm of said electrodes thereby leaving 2 mm of exposed electrode at the tip distal the device housing.

9. The device of claim 8 wherein the electrically non-conductive sheath is a single piece housing the electrode set.

10. The device of claim 7 wherein the two electrodes protrude 38 mm from the device housing.

11. The device of claim 1 wherein the electrode set comprises two or more pairs of electrodes.

12. The device of claim 11 wherein the electrodes forming two of more pairs of electrodes each protrude from the device housing, said protrusion being between 2 mm and 50 mm.

13. The device of claim 12 wherein the electrodes protrude 2 mm from the device housing and are useful for interferential electrotherapy treatment of viral outbreaks at easily accessible areas of the body.

14. The device of claim 12 wherein the electrodes protrude between 25 mm and 50 mm from the device housing.

15. The device of claim 14 wherein the electrodes protrude between 25 mm and 50 mm from the device housing and further comprise an electrically non-conductive sheath covering between 23 mm and 48 mm of said electrodes thereby leaving 2 mm of exposed electrode at the tip distal the device housing.

16. The device of claim 15 wherein the electrically non-conductive sheath is a single piece housing the electrode set.

18. The device of claim 14 wherein the electrodes protrude 38 mm from the device housing.

19. The device of claim 1 wherein the microprocessor is pre-programmed with Waveform Parameters for delivering optimal electrotherapy treatment regimens, said Waveform Parameters comprising; pulse frequency, pulse amplitude, pulse width, duration, modulation, current, pulse times and intensity.

20. The device of claim 19 wherein the pre-programmed microprocessor will vary the Waveform Parameters at a specific time and to a specific degree without user intervention.

21. The device of claim 20 wherein the pre-programmed microprocessor makes real time adjustments to the delivered Waveform Parameters to account for sensed resistance at the skin surface, said real time adjustments in Waveform Parameters being such that the received electrotherapy treatment meets the desired degree for said Waveform Parameters.

22. The device of claim 20 wherein the variations in Waveform Parameters are optimized to treat a viral disease.

23. The device of claim 22 wherein the viral disease is a viral disease selected from the group consisting of HSV-1, HSV-2, VZV-2, HCMV, HHV-6, HHV-7, HHV-8 and EBV.

24. The device of claim 20 wherein the Waveform Parameters are programmed to deliver an optimal interferential electrotherapy.

25. The device of claim 1 wherein the electrotherapy treatment comprises millicurrent electrotherapy treatments and microcurrent electrotherapy treatments.

26. A treatment for viral diseases using the device of claim 1.

27. A method for treating viral diseases using an electrotherapy device comprising the steps of:
a. placing an electrode set exposed from a housing of a device on an area of a body having herpes outbreak;
b. activating the device using a switchable power supply to activate a pre-programmed microprocessor and deliver an electrotherapy through the electrode set; and
c. allowing the device to deliver an optimal electrotherapy to the area of the body wherein said device is placed.

28. The method of claim 27 wherein the electrode set is a single pair of electrodes.

29. The method of claim 27 wherein the electrode set is at least two pair of electrodes.

30. The method of claim 29 wherein the electrode set is useful for delivering an interferential electrotherapy.

31. The method of claim 27 wherein the step of activating the device further comprises using the switchable power supply to activate a power source and the pre-programmed microprocessor, thereby delivering an electrotherapy comprising optimal Waveform Parameters to the electrode set.

32. The method of claim 31 wherein the switchable power supply comprises single pole dual throw switches and gapped circuits.

33. The method of claim 31 wherein the power source comprises cell batteries, rechargeable batteries, embedded power sources and telemetric power sources.

34. The method of claim 27 wherein the optimal electrotherapy further comprises delivery of a set of Waveform Parameters comprising; pulse frequency, pulse amplitude, pulse width, duration, modulation, current, pulse times and intensity.

35. The method of claim 34 wherein the pre-programmed microprocessor will vary the set of Waveform Parameters during a treatment, said variations being at a specific time and to a specific degree and being without user intervention.

36. The method of claim 35 wherein the pre-programmed microprocessor makes real time adjustments to the delivered set of Waveform Parameters to account for sensed resistance at the skin surface, said real time adjustments in the set of Waveform Parameters being such that the received electrotherapy treatment meets the desired degree for said set of Waveform Parameters.

37. The method of claim 35 wherein the variations in the set of Waveform Parameters are optimized to treat a viral disease.

38. The method of claim 37 wherein the viral disease is a viral disease selected from the group consisting of HSV-1, HSV-2, VZV-2, HCMV, HHV-6, HHV-7, HHV-8 and EBV.

39. The method of claim 34 wherein the Waveform Parameters deliver an interferential electrotherapy treatment.

40. The method of claim 27 wherein the optimal electrotherapy comprises millicurrent electrotherapy and microcurrent electrotherapy.

41. A pre-programmed microprocessor comprising the instructions for varying a set of waveform parameters to deliver an optimal electrotherapy treatment.

42. The pre-programmed microprocessor of claim 41 wherein the instructions for varying the set of waveform parameters are determined based on the optimal electrotherapy treatment for treating a viral outbreak.

43. The viral outbreak of claim 42 wherein the virus is selected from the group consisting of HSV-1, HSV-2, VZV-2, HCMV, HHV-6, HHV-7, HHV-8 and EBV.

44. The pre-programmed microprocessor of claim 42 wherein the waveform parameters are varied to a precise degree and at a precise time based on optimal electrotherapy treatment for treating a viral outbreak.

45. The pre-programmed microprocessor of claim 41 wherein the waveform parameters are adjusted based on a real time assessment of resistance in the receiving system so that the received electrotherapy treatment is optimal electrotherapy treatment.
